(19) United States
(12) Patent Application Publication
(10) Pub. No.: US 2024/0033232 A1
(43) Pub. Date: Feb. 1, 2024

(54) New use of (R)-B2-agonists in treatment of sepsis and acute respiratory distress syndrome

(71) Applicant: Wen TAN, Foshan City, GuangDong province (CN)

(72) Inventor: Wen TAN, Foshan City, GuangDong province (CN)

(21) Appl. No.: 18/275,872

(22) PCT Filed: Mar. 10, 2021
(86) PCT No.: PCT/US21/21642

§ 371 Date: Aug. 5, 2023

(51) Int. Cl. A61K 31/137; A61K 31/27; A61K 31/4704; A61K 31/24; A61K 31/44; A61K 31/538; A61K 31/55

(52) U.S. Cl. CPC A61K 31/137; A61K 31/27; A61K 31/4704; A61K 31/24; A61K 31/44; A61K 31/538; A61K 31/55

(57) ABSTRACT

This invention disclosed new use of (R)-enantiomer β2-agonists such as (R)-terbutaline and (R)-salbutamol in treatment of sepsis, sepsis related multiple organ failure and acute respiratory destress syndrome.

The present application claims priority to U.S. Provisional Patent Application No. 62/844,398, filed 7 May 2019, which are herein incorporated by reference in their entirety.

### FIELD OF INVENTION

This invention disclosed a new use of (R)-β2-agonists such as (R)-terbutaline and (R)-salbutamol in treatment of sepsis and sepsis related Acute Respiratory Distress Syndrome (ARDS). This invention disclosed beneficial effects of (R)-β2-agonists such as (R)-terbutaline and (R)-salbutamol in normalizing or ameliorating the dysregulated inflammatory response, in inhibiting of over-activation of both innate and acquired immune-responses, in reducing the over-production of cytokines and chemokines. This invention disclosed protective effects of (R)-β2-agonists on multiple-organs and tissues against sepsis and systemic inflammatory response syndrome. This invention also disclosed the (S)-enantiomer β2-agonists can further worsen the sepsis and relative symptoms. (S)-β2-agonists have the opposite effects as (R)-β2-agonists.

### BACKGROUND OF INVENTION

Sepsis is a clinical syndrome that complicates severe infection and is characterized by the systemic inflammatory response syndrome (SIRS), immune dysregulation, microcirculatory derangements, and end-organ dysfunction including acute respiratory distress syndrome (ARDS) and multiple organ failure. In addition, SIRS can be triggered by a variety of noninfectious conditions, such as trauma, burns, hemorrhagic or hypovolemic shock, pancreatitis, and other disease states. Sepsis can be caused by infections of bacteria, virus and fungi. bacteremia. In these micro-organisms, there are unique groups of molecules which are not associated with human cells. These unique microbial molecules are called pathogen-associated molecular patterns or PAMPs. Examples of microbial-associated PAMPs include: lipopolysaccharide (LPS) from the outer membrane of the Gram-negative cell wall, bacterial lipoproteins and lipopeptides; double-stranded viral RNA unique to many viruses in some stage of their replication and single-stranded viral RNA from many viruses having an RNA genome.

The host response to an infection is initiated when innate immune cells, particularly macrophages, recognize and bind to microbial components. This could occur via the ligation of pathogen-associated molecular patterns (PAMPs) of microorganisms and Pattern recognition receptors (PRRs) such as toll-like receptors (TLRs) on the surface of host immune cells. The Stimulation of PRRs (such as TLRs) by PAMPs triggers the down-stream pathways which will ultimately lead to the activation of mitogen-activated protein kinase (MAPK), nuclear facto(R)-κB (NF-κB) and interferon regulatory factors 3/7 (IRF3/7), which further leads to transcriptional changes and the secretion of inflammatory cytokines, IFNa,b,r; IL-1β, IL-6,9,10,1,215; MCP1, TNFa, chemokines and antimicrobial peptides which together function to target and kill pathogen.

Sepsis develops when the initial, appropriate host response to an infection becomes amplified and subsequently dysregulated, leading to an imbalance between pro-inflammatory and anti-inflammatory responses. In particular, the activation of innate immune response, the “first line of cellular defense”, results in an excessive release of cytokines, chemokines, and other inflammatory regulators from local infection cite to systemic circulation. Furthermore, a dysregulated cytokine release may lead to endothelial dysfunction, characterized by vasodilation and increased capillary permeability. The resulting leakage syndrome is clinically associated with hypotension, macromolecular extravasation, and edema. The dysfunctional epithelial barriers enable pathogens and their products to further invade the host organism, to disturb regulatory mechanisms, and ultimately, to cause remote organ dysfunctions and failure.

Traditionally, sepsis was viewed as an excessive systemic pro-inflammatory reaction to invasive microbial pathogens. More recently, it has been proposed that the early phase of hype(R)-inflammation is followed or overlapped by a prolonged state of immunosuppression, referred to as sepsis induced immunoparalysis. This immunoparalytic states characterized by impaired innate and adaptive immune responses, and may play a central role in the pathogenesis of tissue damage, multiple organ failure, and death induced by sepsis.

The mortality rate from sepsis is approximately 40% in adults, and 25% in children, Septic shock is the 13th leading cause of death in the United States, and the number one cause of deaths in intensive care units. Severe Sepsis leads to multiple organs damage and lung is the most vulnerable organ during sepsis. The acute respiratory distress syndrome (ARDS) occurs in 25% to 50% of patients with sepsis. There is a high mortality rate in patients with ARDS,

Despite the high mortality rate, there are no reliable and safety therapeutically methods or medicine are available to combat sepsis and multiple organ failure.

Corticosteroids, the immune-inhibitor, is one of the major medicine has being used in treatment of severe sepsis. However, the real clinic benefits of corticosteroid have not been confirmed. There may be a small reduction in mortality but also could results in severe adverse effects.

The β-adrenergic system is a well-known effective modulator of the immune system Lymphoid organs such as the spleen, thymus, lymph nodes and bone marrow are predominantly innervated by the sympathetic system. The majority of lymphoid cells, natural killer cells and monocytes express both β1 and/or β2 adrenergic receptors on their surface.

However, in prior art, the use of beta receptor agonist or antagonist seems in controversy. It is believed that β1 blockade may reduce platelet aggregation and have favorable role in the septic pro-coagulant state. β1 blockade as well as β2 activation seems to downregulate pro-inflammatory response by modulating the cytokine production profile. On the other hand, studies also suggest β2-blockade will benefit in modulation the systemic catabolic response to sepsis, [Etienne de Montmollin et al., Critical Care 2009, 13:230].

In a multicenter randomized controlled trial, the therapeutic effects of selective beta2 agonist, salbutamol was tested for patients with acute respiratory distress Syndrome (ARDS) caused by sepsis. salbutamol infusion resulted in 11 percent of increase of mortality in comparing with placebo. Both of the ICU mortality and hospital mortality were significantly increased in salbutamol group in comparing with placebo. This study was stopped early (after recruitment of 326 of a planned 1334 patients) due to increased mortality by salbutamol. [Gates, et. al (2013) Beta-Agonist Lung injury Trial-2 (BALTI-2, Health Technology Assessment, Volume 17 (Number 38). ISSN 1366-5278)

Another clinic trial has shown that inhalation of either fluticasone plus salmeterol (long-acting-β2-agonist) or budesonide plus formoterol (long-acting-β2-agonist) were associated with an increased risk of sepsis related respiratory organ dysfunction. Moreover, the treatments also associated with higher risk of several types of infections, including lower respiratory tract infection, genitourinary tract infection and bacteremia. The risk of salmeterol was higher than formoterol [Cheng-Yi Wang et al., AGING 2019, Vol. 11, No. 17].

The above prior art clinic data disclosed that β2-agnosts is not suitable for treatment of sepsis or ARDS. It demonstrated that use of β2-agnosts actually increase the risk of sepsis and ARDS. However, it is prudent to point out that the β2-agonists used in the prior art above were in their racemic forms, which containing both (R)- and S enantiomers. It is known that different enantiomers may have different biological effects. However, this chirality issue for β2-agonists in treatment of sepsis or ARDS has not been studied in prior art.

There is an urgent a need in the art for reliable and safe medicines for sepsis and related disorders such as ARDS and multiple organ failure.

### DETAIL OF INVENTION

This invention disclosed a new use of (R)-β2-agonists, including SABA, such as terbutaline and salbutamol or LABA, such as salmeterol and formoterol, in treatment of sepsis and sepsis related ARDS and multiple organ failure. Sepsis may be caused by pathogen infections: bacteria, virus or fungus. This invention also disclosed a new used of above (R)-β2-agonists in treatment of systemic inflammatory response syndrome (SIRS) which may be caused by trauma, severe burn, cardiac infraction, heart failure, stroke or other non-infectious diseased or symptoms.

This invention disclosed by surprise that the (S)-enantiomer of salbutamol or terbutaline, i.e. (S)-salbutamol and (S)-terbutaline can increase the risk of sepsis or worsen sepsis and sepsis related ARDS. This is in the opposite of their (R)-enantiomers. Therefore, there is clear advantage of use (R)-enantiomer β2-agonists over racemic (R&S enantiomer mixture) in treatment of sepsis and SIRS, In prior art, use of salbutamol, racemic β2-agonists, increase the incidence and risk of sepsis and ARDS, However, this invention disclosed that (R)-salbutamol and (R)-terbutaline were very effective against sepsis and related ARDS. This difference between this invention and prior art is novel and cannot be anticipated by a person in art.

In prior art, as noted above, it was believed β2-agonists can make the sepsis worse. This invention disclosed that this worsening effect of salbutamol on sepsis can be contributed to the (S)-enantiomer which do not have β2-agonist effects and count for half of (racemic) salbutamol. This disclosure by this invention overcomes a long time technical prejudice and it involves an inventive step.

Sepsis could be the results of dysregulated pro-inflammatory response which lead to a significant systemic increase in inflammatory cells. This invention disclosed that treatment of (R)-salbutamol and (R)-terbutaline can significantly inhibite this increase of inflammatory cells, such as WBC, neutrophils and monocytes and restore them toward a normal level. However, when the (S)-terbutaline was used, the inflammatory cells were further increased from sepsis condition.

Macrophage activation and proliferation was considered to be the key step of initiation and development of sepsis. In one embodiment, LPS (PAMPs from bacteria) induced a proliferation and polarization of macrophages from the peritoneal fluid. While the number of these macrophages significantly reduced toward normal level when treated with (R)-salbutamol. There was also significant reduction of polarized M1 macrophages or M2 macrophages in comparison of sepsis condition. These inhibitory effects can be blocked by using specific β2-recepter blocker.

On the other hand, treatment of sepsis with (S)-salmeterol, both M1 and M2 macrophages were further increased in comparison of sepsis condition.

The ligation of LPS and TLRs will further activate NF-κB and MARK pathways, which will lead to an over-production of cytokines. This invention disclosed that (R)-terbutaline significantly inhibited both of NF-κB and MARK pathways activated by LPS in sepsis, by reducing their expression or phosphorylation.

However, in the contrary, (S)-terbutaline further enhanced both NF-κB and MARK pathways in sepsis. The “cytokine storm” in sepsis involves in excessive release of INFs, IL-1B, IL-2,4,6,10,12, TNFa and MCP etc. This invention disclosed that (R)-terbutaline as well as (R)-salbutamol can effectively prevent or ameliorate the “cytokine storm” and significantly reduced the production and release of these cytokines. In sepsis, there are also over productions of chemokines such as ROS.

This invention disclosed that (R)-terbutaline and (R)-salbutamol could significantly prevent and inhibit the over production of inducible NO (iNO) and another ROS induced by LPS sepsis. In addition, they also prevent the coagulating system disorder such as disseminated intravascular coagulation (DIC) induced by LPS sepsis. On the other hand, their (S)-enantiomer showed opposite effects.

In sepsis, there were severe inflammatory infiltration, edema, lesions and apoptosis in the tissue of lung, heart or kidney or other organs under pathological examination. This invention disclosed that these pathological changes induced by sepsis were greatly ameliorated and improved by treatment of (R)-terbutaline of (R)-salbutamol. However, these pathological changes were further worsened after treatment of their (S)-enantiomer, (S)-terbutaline of (S)-salbutamol.

In sepsis, the dysregulation of inflammatory response results in injury and function loss multiple organs. Acute respiratory distress symptoms (ARDS) is one of the early and common symptoms in sepsis. This invention disclosed that (R)-terbutaline treatment could prevent or restored the deterioration of lung function in sepsis and maintain a normal arterial oxygenation. In earlier sepsis, bronchioles become hyper-responsiveness to bronchial constriction agents such as methacholine (a non-hydrolysable acetylcholine), inflammatory cytokines and chemokines such as histamine. This hyper-responsiveness may contribute to the onset of ARDS. This invention disclosed that bronchial hyper-responsiveness induced by LPS sepsis were significantly ameliorated by the treatment of (R)-terbutaline. While the hyper-responsiveness was further worsened by (S)-terbutaline. In clinic, (R)-enantiomer β2-agonists such as (R)-terbutaline were used as bronchodilator by relaxing the bronchial smooth muscles. This invention disclosed that β2-agonists such as (R)-terbutaline can also be used to ameliorate ARDS by ameliorate hyper-responsiveness of bronchioles to cytokines or chemokines. Furthermore, this invention disclosed that (S)-enantiomer β2-agonists such as (S)-terbutaline can further worsen bronchial hyper-responsiveness, and worsen the ARDS.

In addition, this invention also disclosed a similar effects of other (R)-enantiomer β2-agonists. These disclosures above by this invention are novel and cannot be anticipated by a person in art.

Sepsis has a high mortality rate. In this invention, we disclosed that sepsis by high doses LPS challenge could result in 100% mortality of mice. However, the mortality was greatly reduced and most of animal survived when (R)-terbutaline or (R)-salbutamol were used.

This invention disclosed that other (R)-β2-agonists, including bitolterol, fenoterol, isoprenaline, orciprenaline ormetaproterenol, pirbuterol, procaterol, ritodrine; clenterol and Long-acting β2 agonists: bambuterol, salmeterol arformoterol, Formoterol, Perforomist; trantinterol and Ultra-long-acting β2agonists: abediterol, carmoterol, indacaterol, olodaterol, vilanterol, isoxsuprine, mabuterol and zilpaterol and etc. also had the similar effects as (R)-terbutaline or (R)-salbutamol in reducing the mortality of mice, in inhibiting the excessive release of cytokines or chemokines during sepsis and in ameliorating the ARDS as well as dysregulated inflammatory responses during sepsis. These disclosures have not been disclosed by prior art.

This invention disclosed that the (R)-β2-agonists can be prepared in a pharmaceutical formula including solid dose forms, gel, suppository, liquid or lyophilizes powder for injections, ointment or patches for topic use and aerosol or dry powders for inhalation into lung and nasal.

The invention disclosed that the above (R)-β2-agonists can be administrated via oral, lung inhalation or intravenously injection or infusion, or via an artificial respirator or an extracorporeal membrane oxygenation (ECMO) apparatus.

This inversion disclosed that the above (R)-β2-agonists can be prepared into various pharmaceutical acceptable salts as needed, which including: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

1. The method of use sufficient optic pure (R)-enantiomer β2 agonists and their pharmaceutical acceptable salts in manufacture of pharmaceutical medicaments for treatments of sepsis and sepsis induced multiple organ failure and related syndromes.

2. The method of claim 1, wherein the said related syndromes are acute respiratory distress syndrome (ARDS) and systemic inflammatory response syndrome (SIRS)

3. The method of claim 2, wherein the said acute respiratory distress syndrome (ARDS), is characterized by lung injury, lung inflammation and reduced lung function or bronchial hyper-responsiveness as results of sepsis.

4. The method of claim 1, wherein the said multiple organ failure are lung failure, heart failure and fatal arrhythmia, kidney failure, hepatic failure, CNS dysfunction and cognition deficiency, coagulating system disorder and metabolic disorders as the results of sepsis.

5. The method of claim 1, wherein the said (R)-enantiomer β2 agonists have enantiomer excess value of 85%-98%.

6. The method of claim 1, wherein the said (R)-enantiomer β2-agonists have enantiomer excess value of 98%-99.9%.

7. The method of claim 1, wherein the (R)-enantiomer β2-agonists is terbutaline, salbutamol, bambuterol, vilanterol, clenterol, salmeterol, arformoterol, trantinterol and Indacaterol.

8. The method of claim 1, wherein the said (R)-enantiomer β2-agonists are R or R′R′enantiomers of short-acting β2-agonists: bitolterol, fenoterol, isoprenaline, orciprenaline, ormetaproterenol, pirbuterol, procaterol, ritodrine; and long-acting β2-agonists: formoterol, perforomist; and ultra-long-acting β2-agonists: abediterol, carmoterol, indacaterol, Olodaterol, vilanterol, isoxsuprine, mabuterol and zilpaterol.

9. The method of claim 1, wherein, the said systemic inflammatory response syndrome (SIRS) is the results of pathogen infection or non-pathogen infection including: trauma, severe burn, heart failure, CNS injury, use of antibodies or biological medicine.

10. The method of claim 1, wherein, the said sepsis is the results of pathogen infection including bacteria, virus and fungus.

11. The method of claim 1, wherein, the said sepsis is characterized by an activation and over-proliferation of macrophages and other inflammatory cells.

12. The method of claim 1, wherein, the said sepsis is characterized by either hyper-immuno-inflammation or immunosuppression, or both.

13. The method of claim 1, wherein, the said sepsis is characterized by an excessive release of cytokines, chemokines, ROS and other inflammatory regulators in systemic circulation.

14. The method of claim 1, wherein, the said sepsis or systemic inflammatory response syndrome (SIRS) are characterized by activation of NF-κB and MARK pathways.

15. The method of claim 1, wherein, the pharmaceutical medicaments are solid dose forms, gel, suppository, liquid or lyophilizes powder for injections, ointment or patches for topic use and aerosol or dry powders for inhalation into lung and nasal.

16. The method of claim 1, wherein, the said treatments are characterized by administration of the said medicaments via lung inhalation or intravenously or via an artificial respirator or an extracorporeal membrane oxygenation apparatus.

17. The method of claim 1, wherein, the said pharmaceutical acceptable salts are hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.
